(19) United States
(12) Patent Application Publication
(10) Pub. No.: US 2024/0301338 A1
(43) Pub. Date: Sep. 12, 2024

(54) GEL-ENCAPSULATED CELL PRODUCTION APPARATUS AND CELL CULTURE SYSTEM

(71) Applicants: Canon Medical Systems Corporation, Otawara-shi (JP); CANON KABUSHIKI KAISHA, Tokyo (JP)

(72) Inventor: Kenta KAMINO, Utsunomiya (JP)

(73) Assignees: Canon Medical Systems Corporation, Otawara-shi (JP); CANON KABUSHIKI KAISHA, Tokyo (JP)

(21) Appl. No.: 18/596,854

(22) Filed: Mar. 6, 2024

(30) Foreign Application Priority Data
Mar. 8, 2023 (JP) 2023-035787

(51) Int. Cl. C12M 1/12; C12M 1/06; C12M 1/34; C12M 1/42; C12M 3/00; C12N 5/071; C12N 5/077; C12N 5/0775

(52) U.S. Cl. CPC C12M 25/01; C12M 23/48; C12M 27/06; C12M 35/02; C12M 41/12; C12M 41/36; C12N 5/0625; C12N 5/0656; C12N 5/0665; C12N 2533/74; C12N 2533/80

(57) ABSTRACT

A gel-encapsulated cell production apparatus according to an embodiment has a first droplet generator, a second droplet generator, and a gel-encapsulated cell generator. The first droplet generator generates a first droplet in which a processed cell is encapsulated. The second droplet generator generates a second droplet in which a gelator is encapsulated. The gel-encapsulated cell generator is connected to the first droplet generator and the second droplet generator via a channel, and the gel-encapsulated cell generator blends the first droplet and the second droplet and generates a gel-encapsulated cell, which is the cell encapsulated in a gel originating in the gelator.

### CROSS-REFERENCE TO RELATED APPLICATION (S)

This application is based upon and claims the benefit of priority from Japanese Patent Application No. 2023-035787, filed Mar. 8, 2023, the entire contents of which are incorporated herein by reference.

### FIELD

Embodiments described herein relate generally to a gel-encapsulated cell production apparatus and a cell culture system.

### BACKGROUND

Cell processing, such as transfection, which introduces nucleic acids, etc., into cells, has been performed. Lipoplexes, etc., are adopted as the technique of transfection, but they are known to exhibit low introduction efficiency for floating cells. There is known a technology of encapsulating a cell and a transfection reagent in a microreactor of a droplet using microfluidic technology and thereby achieving higher efficiency (hereinafter referred to as “technology 1”), as compared to bulk transfection. Also known is a technology of encapsulating a cell in a droplet using a microchannel fluidic device, and then causing the droplet to gelate and culturing the cell in the gel droplet (hereinafter referred to as “technology 2”). Gelation in a droplet can be promoted by, for example, also encapsulating Ca-NTA (nitrilotriacetic acid), which chelates with Ca²⁺, in the droplet, utilizing the phenomenon in which alginic acid is caused to gelate by Ca²⁺, and causing the oil containing the acid to react with the droplet, utilizing the property of Ca-NTA releasing Ca²⁺ in a low-pH environment.

The technology 1 and the technology 2 can be combined as a method of culturing cells one by one after performing transfection in the droplet. However, a technique such as lipofection that introduces a complex formed of a lipid having a positive charge and a substance such as a nucleic acid having a negative charge into a cell may decrease the efficiency of introduction into the cell due to the electrostatic characteristics of materials such as sodium alginate and sodium hyaluronate that are generally used for gelation. Also, the high viscosity of the gelator makes agitation inefficient, which may reduce the number of collisions between the cell and the transfection reagent, thereby decreasing the introduction efficiency.

### BRIEF DESCRIPTION OF THE DRAWING (S)

FIG. 1 shows an exemplary configuration of a gel-encapsulated cell production system according to a first embodiment.

FIG. 2 schematically shows the exemplary configuration of a gel-encapsulated cell production apparatus shown in FIG. 1.

FIG. 3 schematically shows an exemplary structure of a first droplet generator shown in FIG. 2.

FIG. 4 schematically shows an exemplary structure of a second droplet generator shown in FIG. 2.

FIG. 5 schematically shows an exemplary structure of a gel-encapsulated cell generator shown in FIG. 2.

FIG. 6 shows an exemplary configuration of a control apparatus of the gel-encapsulated cell production system shown in FIG. 1.

FIG. 7 shows a process procedure of producing a gel-encapsulated cell using the gel-encapsulated cell production system according to the first embodiment.

FIG. 8 schematically shows the step of generating a first droplet (SA2) shown in FIG. 7.

FIG. 9 schematically shows the step of generating a second droplet (SA5) shown in FIG. 7.

FIG. 10 schematically shows the steps of generating a gel-encapsulated cell (SA6 to SA8) shown in FIG. 7.

FIG. 11 schematically shows mechanoporation using a narrowed channel according to a modification 2.

FIG. 12 shows an exemplary configuration of a cell culture system according to a second embodiment.

FIG. 13 shows a process procedure of culturing a gel-encapsulated cell using the cell culture system according to the second embodiment.

### DETAILED DESCRIPTION

A gel-encapsulated cell production apparatus according to an embodiment includes: a baseplate; a first droplet generator that is arranged on the base plate and generates a first droplet in which a processed cell is encapsulated; a second droplet generator that is arranged on the baseplate and generates a second droplet in which a gelator is encapsulated; and a gel-encapsulated cell generator that is arranged on the baseplate, is connected to the first droplet generator and the second droplet generator via a channel, blends the first droplet and the second droplet, and generates a gel-encapsulated cell which is the cell encapsulated in a gel originating in the gelator.

Hereinafter, a gel-encapsulated cell production apparatus and a cell culture system according to embodiments will be described in detail with reference to the accompanying drawings.

### First Embodiment

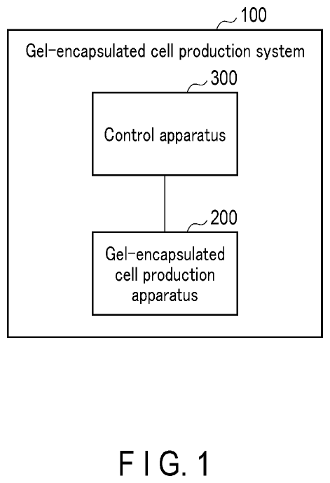

FIG. 1 shows an exemplary configuration of a gel-encapsulated cell production system 100 according to a first embodiment. As shown in FIG. 1, the gel-encapsulated cell production system 100 is a mechanical system that produces a processed cell encapsulated in a gel (hereinafter referred to as “a gel-encapsulated cell”). The gel-encapsulated cell production system 100 includes a gel-encapsulated cell production apparatus 200 and a control apparatus 300. The gel-encapsulated cell production apparatus 200 is a base-form apparatus on which various structures for producing a gel-encapsulated cell are formed. The control apparatus 300 is a machine that performs ejection and collection of various substances to and from the gel-encapsulated cell production apparatus 200, control of the various devices, etc.

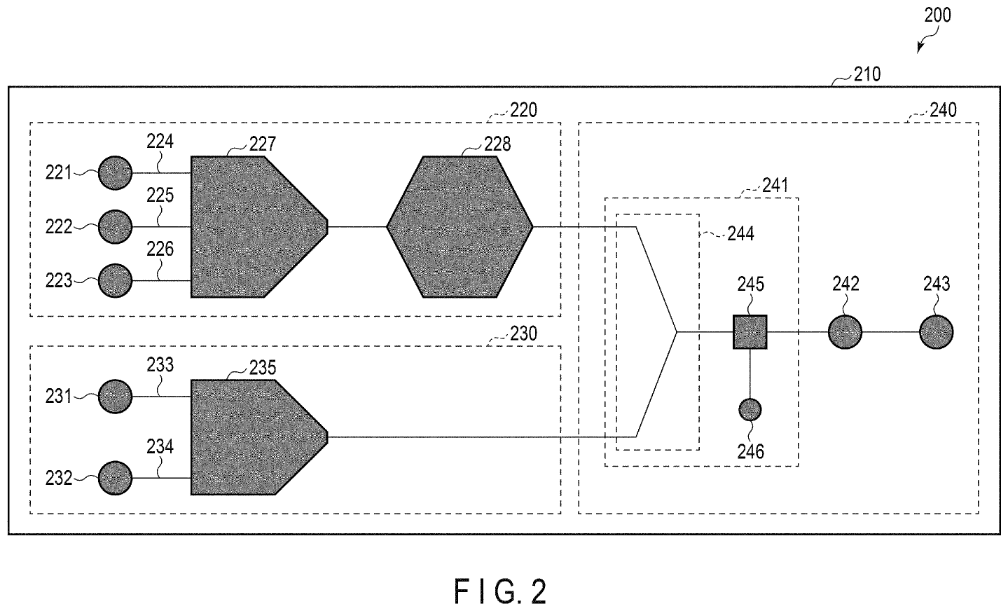

FIG. 2 schematically shows an exemplary configuration of the gel-encapsulated cell production apparatus 200. The gel-encapsulated cell production apparatus 200 is realized by a microfluidics channel chip having a series of structures of processing a population of cells one by one and encapsulating the cells directly into gels. Specifically, the gel-encapsulated cell production apparatus 200 has a base 210, a first droplet generator 220, a second droplet generator 230, and a gel-encapsulated cell generator 240, as shown in FIG. 2.

The base 210 is a plate-shaped structure that includes a glass, a silicone and/or a resin, etc., as a material. In the embodiment, the base 210 is formed of, for example, a phenol resin, an epoxy resin, an acrylic resin, or a silicone resin. More specifically, PDMS (dimethylpolysiloxane) or COP (cycloolefinpolymer) may be used. The first droplet generator 220, the second droplet generator 230, and the gel-encapsulated cell generator 240 are formed on the base 210 through microfabrication that applies photolithography.

The first droplet generator 220 is arranged on the baseplate 210 and generates a first droplet in which a processed cell is encapsulated. As an example, the first droplet generator 220 mixes a cell and a processing reagent for processing the cell to generate a first droplet. The “cell” is a target to be processed and cultured. The type of cell is not particularly limited, and any type of cell can be used according to the purpose. The term “processing” means genetic transformation (transfection) such as gene introduction and genome editing. Gene introduction introduces a foreign substance into the cell and transforms a partial trait of the cell into a trait of the foreign substance. Genome editing introduces a genome editing protein (CRISPR-Cas9, etc.), which is a foreign substance, mRNA, and gRNA, into the cell to cause mutation of a partial trait of the cell.

As an example, a combination of a viral vector, a plasmid vector, lipofection, a liposome (lipofectamine, etc.), a protein, an RNA (mRNA, miRNA, siRNA, etc.), a low-molecular compound, a nanoparticle, a cationic polymer, a genome editing protein (CRISPR-Cas9), and gRNA can be used as the foreign substance to be introduced into the cell. A biological method, a chemical method, a physical method, etc., are available for transfection. The biological method for transfection can be a method that uses a viral vector. The chemical method for transfection can be a method that uses a polyplex, a lipoplex, etc. The physical method for transfection can be a method such as sonoporation, electroporation, mechanoporation, magnetofection, etc. The processing reagent is a reagent that contains the above foreign substances and causes transfection of the cell.

The first droplet generator 220 has a cell entering section 221, a processing reagent entering section 222, a first oil entering section 223, a cell channel 224, a processing reagent channel 225, a first oil channel 226, a droplet generator 227, and a promoter 228. The cell entering section 221 is a place where a cell is dropped. The processing reagent entering section 222 is a place where a processing reagent is dropped. The first oil entering section 223 is a place where a first oil is dropped. An oil or a surfactant is used as the first oil. An organic oil such as a fluorinated oil or a mineral oil, which are not toxic to cells, is preferably used. The cell channel 224 is a channel through which the cell delivered to the cell entering section 221 passes. The processing reagent channel 225 is a channel through which the processing reagent dropped in the processing reagent entering section 222 passes. The first oil channel 226 is a channel through which the first oil dropped in the first oil entering section 223 passes.

The droplet generator 227 converges the cell channel 224, the processing reagent channel 225, and the first oil channel 226, and generates a third droplet in which the cell and the processing reagent are encapsulated using the first oil. Specifically, the droplet generator 227 is realized by the joint channel formed of the cell channel 224, the processing reagent channel 225, and the first oil channel 226. The three types of channels, the cell channel 224, the processing reagent channel 225, and the first oil channel 226, may be joined together in one place. Alternatively, the cell channel 224 and the processing reagent channel 225 may be joined together, and the joint channel formed thereof and the first oil channel 226 may be joined together. The promoter 228 has a structure and/or a device for promoting the cell processing performed using the processing reagent inside the third droplet.

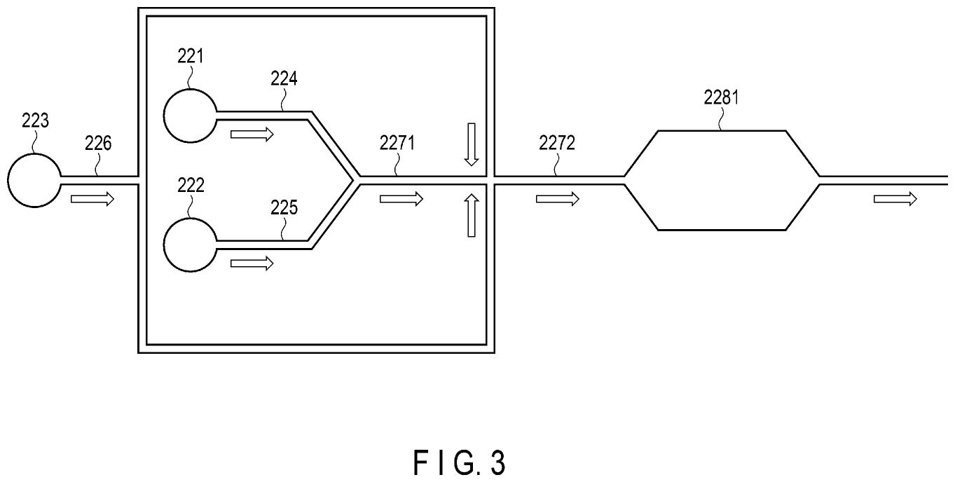

FIG. 3 schematically shows an exemplary structure of the first droplet generator 220. As shown in FIG. 3, the cell channel 224 is connected to the cell entering section 221, and the processing reagent channel 225 is connected to the processing reagent entering section 222. The cell channel 224 and the processing reagent channel 225 are joined together to form a first joint channel 2271. The first oil channel 226 is connected to the first oil entering section 223, and the first oil channel 226 is branched into two so as to surround the cell entering section 221, the processing reagent entering section 222, the cell channel 224, the processing reagent channel 225, and the first joint channel 2271, and joins the first joint channel 2271 to form a second joint channel 2272. The width, depth, length, shape, etc., of the various channels 224, 225, 226, 2271, and 2272 may be freely set.

In the first joint channel 2271, the cell from the cell channel 224 and the processing reagent from the processing reagent channel 225 are joined together. In the second joint channel 2272, the cell, the processing reagent, and the first oil are joined together, so that the third droplet in which the cell and the processing reagent are encapsulated is generated by the first oil. A container 2281 for holding the third droplet (hereinafter referred to as a “processing promoting container 2281”) as the promoter 228 is connected to the second joint channel 2272. The temperature of the processing promoting container 2281 is adjusted by a temperature adjuster (not shown) to a temperature that promotes the cell processing performed by the use of the processing reagent. The first droplet is generated by the processing of the cell inside the third droplet using the processing reagent. The width, depth, capacity, shape, etc., of the processing promoting container 2281 may be freely set.

As shown in FIG. 2, the second droplet generator 230 is arranged on the baseplate 210 and generates a second droplet in which a gelator is encapsulated. For example, alginic acid, hyaluronic acid, gelatin, agarose, PNIPAAm, PEG, PVA, chitosan, derivatives of these substances, derivatives of these substances that include a laminin motif, and derivatives of these substances that have a side chain that functions to gelate upon addition of a crosslinker and stimulation with light, heat, etc., can be used as the gelator. The gelator to be used can be freely selected from among these examples of the gelator according to the cell used and the cell processing method adopted.

The second droplet generator 230 has a gelator entering section 231, a second oil entering section 232, a gelator channel 233, a second oil channel 234, and a droplet generator 235. The gelator entering section 231 is a place to which a gelator is delivered. The second oil entering section 232 is a place to which a second oil is delivered. The gelator channel 233 is a channel through which the gelator delivered to the gelator entering section 231 passes. The second oil channel 234 is a channel through which the second oil dropped in the second oil entering section 232 passes. The droplet generator 235 converges the gelator channel 233 and the second oil channel 234, and generates a second droplet in which the gelator is encapsulated by using the second oil. Specifically, the droplet generator 235 is realized by the joint channel formed of the gelator channel 233 and the second oil channel 234.

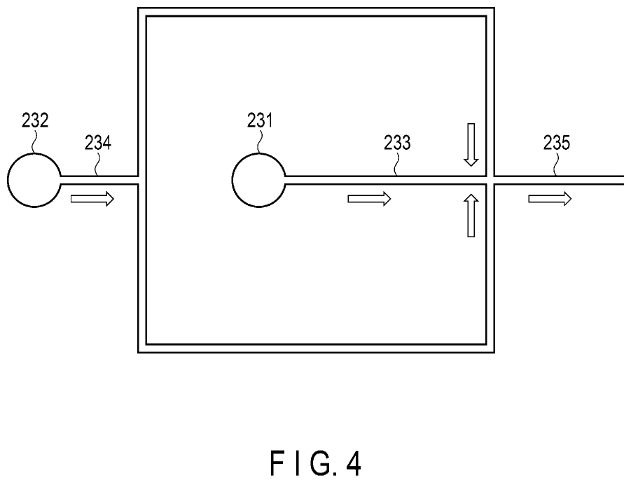

FIG. 4 schematically shows an exemplary structure of the second droplet generator 230. As shown in FIG. 4, the gelator channel 233 is connected to the gelator entering section 231, the second oil channel 234 is connected to the second oil entering section 232, and the second oil channel 234 is branched into two so as to surround the gelator entering section 231 and the gelator channel 233, and joins the gelator channel 233 to form the joint channel 235. The second oil used may have the same composition as that of the first oil. In the joint channel 235, the second droplet in which the gelator is encapsulated is generated by the second oil. The width, depth, length, shape, etc., of the various channels 233, 234, and 235 may be freely set.

As shown in FIG. 2, the gel-encapsulated cell generator 240 is arranged on the baseplate 210, is connected to the first droplet generator 220 and the second droplet generator 230 via a channel, blends the first droplet and the second droplet, and generates a gel-encapsulated cell, which is the cell encapsulated in a gel originating in the gelator. Specifically, the gel-encapsulated cell generator 240 has a blender 241, a stirrer 242, and a stimulus introducer 243.

The blender 241 blends the first droplet delivered from the first droplet generator 220 and the second droplet delivered from the second droplet generator 230. A droplet obtained after the blending of the first droplet and the second droplet is called a blended droplet. That is, the blender 241 generates a blended droplet. Specifically, the blender 241 has a joint section 244, a holding container 245, and an electrode 246. The joint section 244 has a channel where the channel through which the first droplet passes and the channel through which the second droplet passes are joined together. The holding container 245 is a container that is connected to the joint section 244 and holds the first droplet and the second droplet. In the holding container 245, the first droplet and the second droplet are held close to each other. The electrode 246 applies an electric field for inducing the blending to the first droplet and the second droplet held in the holding container 245. The material of the electrode 246 is not particularly limited; however, for example, titanium (Ti), gold (Au), etc., are used.

The stirrer 242 has a structure and/or a device for stirring the gelator and the processed cell inside the blended droplet. The stimulus introducer 243 has a structure and/or a device for giving the blended droplet a stimulus for inducing the gelation of the cell with the gelator in order to generate a gel-encapsulated cell. The stimulus for inducing the gelation is a gelator-dependent reagent. As an example, the reagent includes a crosslinker, an acid, or an ion. The reagent is provided in the form of being solved in an oil.

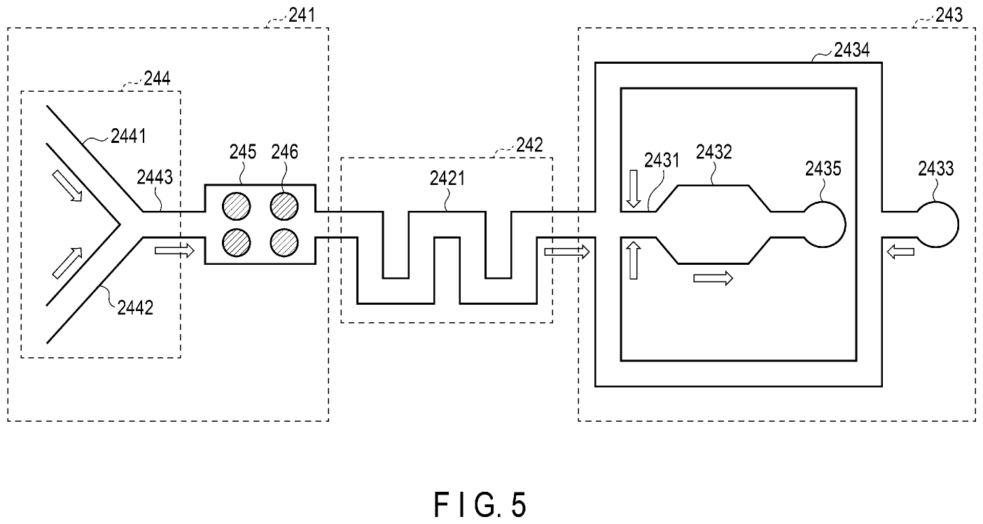

FIG. 5 schematically shows an exemplary structure of the gel-encapsulated cell generator 240. As shown in FIG. 5, the joint section 244 has a channel 2441 through which the first droplet delivered from the first droplet generator 220 passes, a channel 2442 through which the second droplet delivered from the second droplet generator 230 passes, and a joint channel 2443 where the channel 2441 and the channel 2442 are joined together. The joint channel 2443 is connected to the holding container 245. In the holding container 245, the first droplet and the second droplet are held close to each other. The holding container 245 is provided with multiple electrodes 246 for applying an electric field to the first droplet and the second droplet held in the holding container 245. The electrodes 246 may be arranged in any position, provided that the electrodes 246 can apply an electric field to the first droplet and the second droplet held in the holding container 245. The application of an electric field blends the first droplet and the second droplet, generating the blended droplet.

As shown in FIG. 5, the stirrer 242 has a channel 2421 for stirring the processed cell and the gelator inside the blended droplet (hereinafter, this channel is referred to as a “stirring channel 2421”). The inlet port of the stirring channel 2421 is connected to the holding container 245. The stirring channel 2421 is not linear but zig-zags from its inlet port to its outlet port. With the stirring channel 2421 zig-zagging, it is possible to stir the processed cell and the gelator inside the blended droplet.

As shown in FIG. 5, the stimulus introducer 243 has a joint channel 2431, a holding container 2432, a third oil entering section 2433, a third oil channel 2434, and a gel-encapsulated cell collecting point 2435. The joint channel 2431 is a channel where the stirring channel 2421 and the third oil channel 2434 are joined together. The holding container 2432 is a container that is connected to the joint channel 2431 and holds the blended droplet and a third oil. The third oil entering section 2433 is a place where the third oil is dropped. The third oil is an oil in which a reagent for inducing the gelation of the processed cell via the gelator is solved. The third oil channel 2434 is a channel through which the third oil dropped in the third oil entering section 2433 passes. The third oil channel 2434 is branched from the third oil entering section 2433 and connected to the joint channel 2431. The third oil is delivered to the joint channel 2431 through the third oil channel 2434. In the holding container 2432, gelation of the processed cell via the gelator contained in the blended droplet is induced by the third oil. Specifically, the water contained in the blended fluid is hardened by the gelator and turns into a gel, so that the processed cell is enclosed in the gel. A gel-encapsulated cell is thereby generated. The gel-encapsulated cell collecting point 2435 is connected to the holding container 2432 and is a place where gel-encapsulated cells are collected.

The width, depth, length, shape, etc., of the various channels 2441, 2442, 2443, 2421, 2431, and 2434 may be freely set. The width, depth, capacity, shape, etc., of the various containers 245 and 2432 may be freely set. The stirring channel 2421 need not be provided if the processed cell can be caused to gelate without the stirring channel 2421.

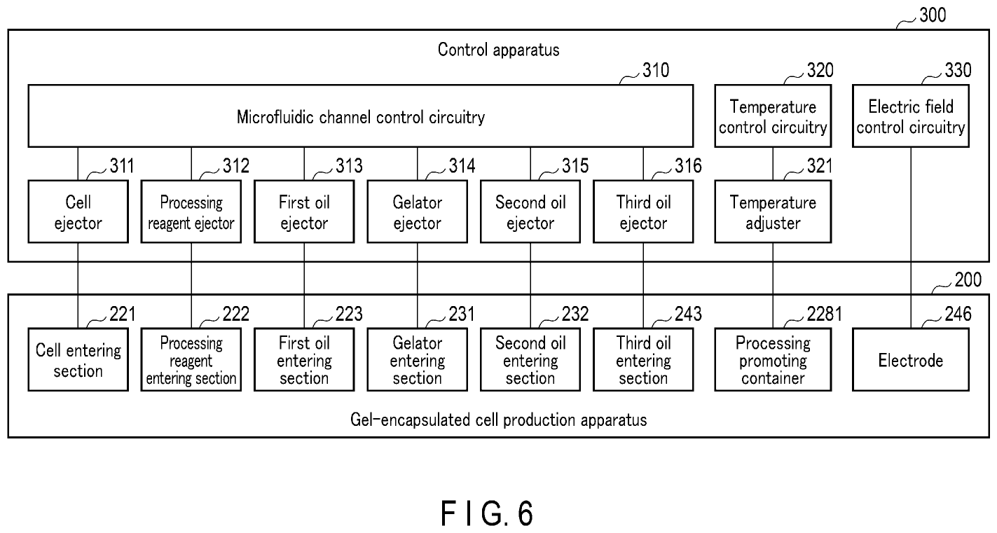

FIG. 6 shows an exemplary configuration of the control apparatus 300 of the gel-encapsulated cell production system 100. As shown in FIG. 6, the control apparatus 300 includes microfluidic channel control circuitry 310, a cell ejector 311, a processing reagent ejector 312, a first oil ejector 313, a gelator ejector 314, a second oil ejector 315, a third oil ejector 316, temperature control circuitry 320, a temperature adjuster 321, and electric field control circuitry 330.

The microfluidic channel control circuitry 310, the cell ejector 311, the processing reagent ejector 312, the first oil ejector 313, the gelator ejector 314, the second oil ejector 315, and the third oil ejector 316 constitute a fluid delivery system. The microfluidic channel control circuitry 310 is control circuitry that synchronously controls the cell ejector 311, the processing reagent ejector 312, the first oil ejector 313, the gelator ejector 314, the second oil ejector 315, and the third oil ejector 316. The cell ejector 311 is a machine that is detachably provided to the cell entering section 221 and ejects the cell to the cell entering section 221. The processing reagent ejector 312 is a machine that is detachably provided to the processing reagent entering section 222 and ejects the processing reagent to the processing reagent entering section 222. The first oil ejector 313 is a machine that is detachably provided to the first oil entering section 223 and ejects the first oil to the first oil entering section 223. The gelator ejector 314 is a machine that is detachably provided to the gelator entering section 231 and ejects the gelator to the gelator entering section 231. The second oil ejector 315 is a machine that is detachably provided to the second oil entering section 232 and ejects the second oil to the second oil entering section 232. The third oil ejector 316 is a machine that is detachably provided to the third oil entering section 2433 and ejects the third oil to the third oil entering section 2433.

Each of the machines, the cell ejector 311, the processing reagent ejector 312, the first oil ejector 313, the gelator ejector 314, the second oil ejector 315, and the third oil ejector 316, has an injector and a pump. The injector is a container having a shape that can contain and eject a solution, and the pump actuates the injector to deliver the solution. The pump actuates the injector under the control executed by the channel control circuitry 310. The channel control circuitry 310 is capable of controlling the pressure and the flow rate of the solution delivered from the injector by adjusting the operation of the pump. A micropump, a syringe pump, etc., can be used as the pump. Examples of the micropump that can be used include a piezoelectric one, a light-driven one, a nanomotor one, a capillary one.

The temperature control circuitry 320 sends an operation signal to the temperature adjuster 321 to control temperature. An electronic thermostat, a mechanical thermostat, or the like is used as the temperature control circuitry 320. Under the control executed by the temperature control circuitry 320, the temperature adjuster 321 adjusts the temperature inside the processing promoting container 2281 to a temperature that promotes cell processing performed using the processing reagent. As an example, a heater or a cooler is used as the temperature adjuster 321.

The electric field control circuitry 330 applies an electric field to the holding container 245 via the electrode 246. The electric field control circuitry 330 has power supply circuitry and control circuitry. The power supply circuitry generates a direct-current voltage or an alternate-current voltage. The control circuitry raises or lowers the voltage generated by the power supply circuitry to a predetermined voltage value and applies it to the electrode 246.

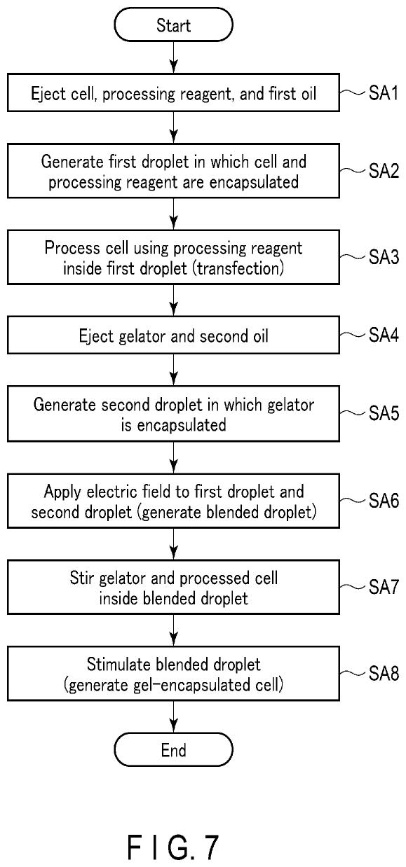

Next, a process of producing a gel-encapsulated cell using the gel-encapsulated cell production system 100 will be described with reference to FIG. 7. Lipofection is performed as the transfection according to the example shown in FIG. 7.

FIG. 7 shows a process procedure of producing a gel-encapsulated cell using the gel-encapsulated cell production system 100. As shown in FIG. 7, the microfluidic channel control circuitry 310 actuates the cell ejector 311 to eject a cell to the cell entering section 221, actuates the processing reagent ejector 312 to eject a processing reagent to the processing reagent entering section 222, and actuates the first oil ejector 313 to eject a first oil to the first oil entering section 223 (step SA1). After step SA1, a first droplet in which the cell and the processing reagent are encapsulated is generated in the promoter 228 (step SA2).

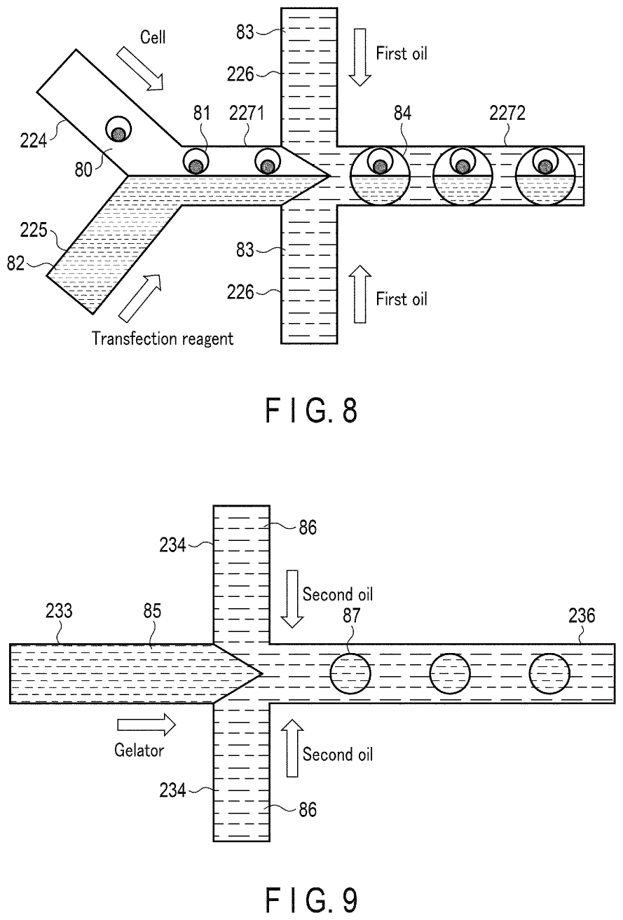

FIG. 8 schematically shows the process of generating a first droplet (SA2). As shown in FIG. 8, a cell fluid 80, which is a fluid containing a cell 81, is delivered to the cell channel 224, a transfection reagent 82, which is a processing reagent, is delivered to the processing reagent channel 225, and a first oil 83 is delivered to the first oil channel 226. As one example, a gene introducing reagent Lipofectamine (registered trademark)+a plasmid vector, etc., is used as the transfection reagent 82. Lipofectamine (registered trademark) and the introduced substance are, for example, mixed together in a vortex, etc., in advance, and a resulting mixture is ejected to the processing reagent entering section 222. As another example, a zig-zagging channel may be provided in a part of the processing reagent channel 225, so that stirring and mixing are performed in the channel.

The first oil 83 is used as a continuous phase for generating a first droplet 84. As an example, a material made by adding a surfactant, such as dSURF (registered trademark) (Fluigent), picoSURF (registered trademark) (Sphere Fluidics), etc., to a fluorinated oil, such as HFE (hydrofluoroether), can be used as the first oil 83. If a mineral oil, a silicone oil, or the like is used, a surfactant that matches the first oil 83 can be used.

As shown in FIG. 8, the cell fluid 80 containing the cell 81 and the transfection reagent 82 are mixed together at the joint channel 2271. The first oil channel 226 joins the joint channel 2271 from the two directions perpendicular to the joint channel 2271, whereby the joint channel 2272 is formed. At the junction of the joint channel 2271 and the first oil channel 226, a mixed fluid made of the cell 81 and the transfection reagent 82 is divided at regular intervals by the first oil 83 evenly delivered from two directions. The first droplet 84 is thereby generated. The first droplet 84 includes the cell 81 contained in the cell fluid 80 and a fluid such as water. The particle size of the first droplet 84 generated is not particularly limited, provided that the first droplet 84 can include one cell 81; however, the particle size is assumed to be about several micrometers to several millimeters. The first droplet 84 is delivered to the promoter 228.

The particle size and the delivery cycle (the time interval for generation) of the first droplet 84 can be freely adjusted by the control of the delivery pressure (s) and/or the flow rate (s) of the cell fluid 80, the transfection reagent 82, and/or the first oil 83 executed by the microfluidic channel control circuitry 310. Typically, the cells 81 are delivered one by one to the cell channel 224 by the cell ejector 311 at regular time intervals. The delivery pressure and/or the flow rate of the first oil 83 is/are adjusted such that only one cell 81 is included in the first droplet 84. Alternatively, the concentration of the cell fluid 80 may be adjusted based on a Poisson distribution such that only one cell 81 is included in the first droplet 84. Alternatively, a mechanism that aligns the cells 81 one by one may be provided to the cell channel 224.

After step SA2, the promoter 228 processes the cell (performs transfection) using a processing reagent inside the first droplet (step SA3). The first droplet generated in step SA2 is retained in the processing promoting container 2281 shown in FIG. 3. In the processing promoting container 2281, the cell is subjected to transfection using a processing reagent inside the first droplet.

If a viral vector, a lipoplex or a polyplex etc is used as transfection, the temperature inside the processing promoting container 2281 is adjusted by the temperature control circuitry 320 in order to promote the transfection. As an example, if a Sendai virus is used as a viral vector, the temperature inside the processing promoting container 2281 is maintained at 37° C. since 37° C. is an optimal temperature. The processing promoting container 2281 may have a zig-zagging channel since the cell and the transfection reagent are stirred inside the first droplet. Stirring the cell and the transfection reagent promotes the transfection performed using Sendai virus.

After step SA3, the microfluidic channel control circuitry 310 actuates the gelator ejector 314 to eject a gelator to the gelator entering section 231, and actuates the second oil ejector 315 to eject a second oil to the second oil entering section 232 (step SA4). After step SA4, a second droplet in which the gelator is encapsulated is generated (step SA5).

FIG. 9 schematically shows the process of generating a second droplet (SA5). As shown in FIG. 9, the gelator is delivered to the gelator channel 233, and the second oil is delivered to the second oil channel 234. The second oil channel 234 joins the gelator channel 233 from the two directions perpendicular to the gelator channel 233, whereby the joint channel 236 is formed. At the junction of the gelator channel 233 and the second oil channel 234, the second oil is delivered at a given delivery pressure and/or a given flow rate, and the gelator is divided at regular intervals by the second oil, whereby a second droplet 87 is generated. The channel control circuitry 310 adjusts the delivery pressures and the flow rates of the gelator 85 from the gelator ejector 314 and the second oil 86 from the second oil ejector 315 such that the delivery cycle of the second droplet 87 coincides with the delivery cycle of the first droplet 84. The second droplet 87 is delivered to the gel-encapsulated cell generator 240.

After step SA5, the electric field control circuitry 330 applies an electric field to the first droplet and the second droplet (step SA6). After step SA6, the gelator and the cell are stirred inside the blended droplet (step SA7). After step SA7, a stimulus is given to the blended droplet (step SA8).

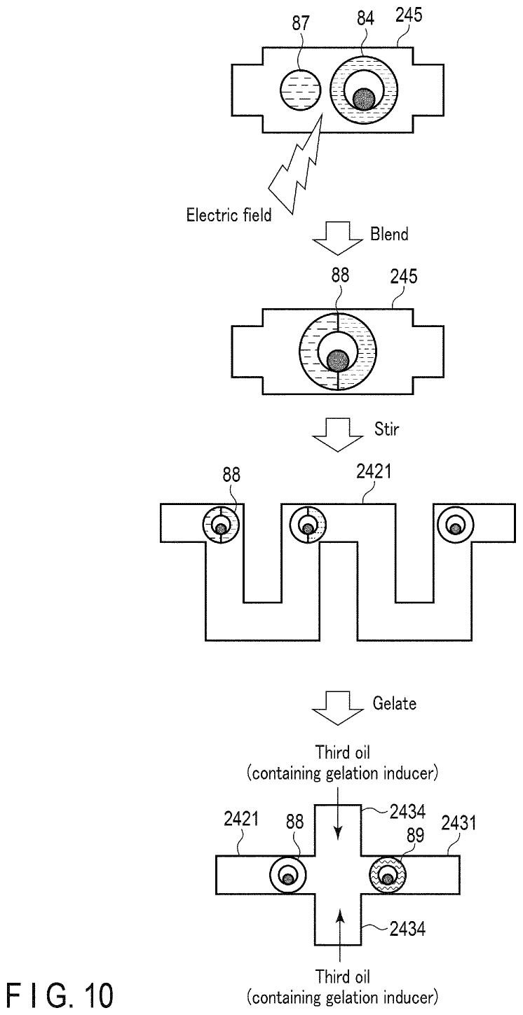

FIG. 10 schematically shows the process of generating a gel-encapsulated cell (SA6 to SA8). In step SA6, the first droplet 84 from the first droplet generator 220 and the second droplet 87 from the second droplet generator 230 are retained close to each other in the holding container 245, as shown in FIG. 10. The processed cell is contained in the first droplet 84, and the gelator is contained in the second droplet 87. The electric field control circuitry 330 applies an electric field to the first droplet 84 and the second droplet 87 via the electrode 246. The application of an electric field blends the first droplet 84 and the second droplet 87, generating a blended droplet 88. It is assumed that immediately after the generation of the blended droplet 88, the processed cell and the gelator are separated from each other inside the blended droplet 88. The blended droplet 88 is delivered to the stirring channel 2421.

Since the stirring channel 2421 zig-zags, the processed cell and the gelator are stirred inside the blended droplet 88 by the blended droplet 88 passing through the stirring channel 2421. The third oil channel 2434 joins the stirring channel 2421. At the junction of the stirring channel 2421 and the third oil channel 2434, a third oil is added to the blended droplet 88. This promotes the gelation of the processed cell inside the blended droplet 88. A gel-encapsulated cell 89 is generated by the encapsulation of the processed cell in the gel.

As an example, if a material that starts gelating via an ion is used, a third oil containing the ion is delivered. For example, alginic acid can be used as such an ion. Alternatively, it is possible to encapsulate a gelator in a droplet together with a chelator having an ion, such as Ca-EDTA or Ca-NTA, and release the ion from the chelator using an acid, thereby promoting the gelation. For example, it is possible to cause gelation by bringing a material made by adding an organic acid such as acetic acid to HTF into contact with the droplet.

The blended droplet is delivered to the holding container 2432 via the joint channel 2431. The blended droplet is retained in the holding container 2432 for a predetermined period of time, causing the gelation reaction to progress. A gel-encapsulated cell is thereby generated. Thereafter, the gel-encapsulated cell is delivered to the gel-encapsulated cell collecting point 2435 and collected by any means.

The explanation of the process of producing a gel-encapsulated cell shown in FIG. 7 will end with the above descriptions. The flow of the process of producing a gel-encapsulated cell shown in FIG. 7 is an example, and the embodiment is not limited thereto. Various modifications can be made, provided that a gel-encapsulated cell can be produced from the first droplet and the second droplet.

### Modification 1

The transfection according to the above embodiment is performed through lipofection; however, transfection according to a modification 1 may be performed through a viral vector. The transfection through a viral vector can be performed in the same manner as the transfection through lipofection. Specifically, a viral vector having any infectivity titer may be used as a processing reagent.

### Modification 2

The transfection according to the above embodiment is performed through lipofection; however, transfection according to a modification 2 may be performed through mechanoporation. In this case, a processing reagent is not particularly limited; however, proteins, nucleic acids, nanoparticles, low-molecular compounds, and middle-molecular compounds such as peptides may be used as a processing reagent. The processing promoting container 2281 according to the modification 2 is constituted by a channel that realizes mechanoporation. As an example, the processing promoting container 2281 is realized by a narrowed channel.

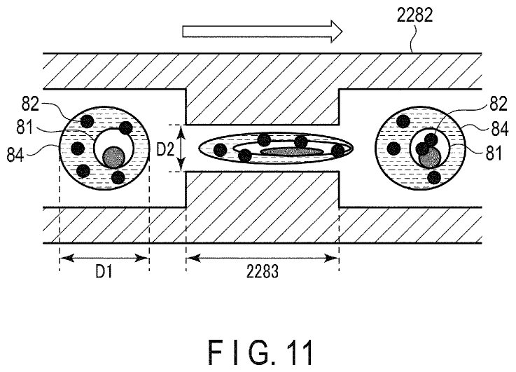

FIG. 11 schematically shows mechanoporation using a narrowed channel 2282. As shown in FIG. 11, the narrowed channel 2282 has a section 2283 having a width D2 smaller than a diameter D1 of the first droplet 84. When the first droplet 84 enters the section 2283, the first droplet 84 is compressed, causing a hole to be generated in the cell 81. Thereafter, the first droplet 84 passes through the section 2283 and is delivered to the gel-encapsulated cell generator 240. The transfection reagent 82 enters the cell 81 through the hole generated in the cell 81. Mechanoporation is thereby achieved.

### Modification 3

In the above embodiment, the first droplet generator 220 processes a cell after encapsulating the cell in a droplet. However, the embodiment is not limited thereto. The first droplet generator 220 according to a modification 3 may encapsulate a processed cell in a droplet. Specifically, the first droplet generator 220 according to the modification 3 processes a cell with a processing reagent and encapsulates the processed cell in a droplet using a first oil, thereby generating a first droplet.

### Modification 4

In the above embodiment, the type of the gel-encapsulated cell produced is not particularly limited. As an example, the embodiment can be applied to production of an iPS cell encapsulated in a gel as a gel-encapsulated cell. In this case, PBMCs (peripheral blood mononuclear cells), hematopoietic stem cells, umbilical cord blood cells, skin cells, or fibroblasts are used as the “cells” to pass through the cell channel 224. PBMCs are mononuclear cells isolated from peripheral blood and include lymphocytes such as T cells, B cells, NK cells, monocytes, and dendritic cells. As the “processing reagent” to pass through the processing reagent channel 2225, either Sendai virus or a combination of a lipofection reagent and a vector is used. Cytotune (ID Pharma Co., Ltd.), SRV (TOKIWA-Bio Inc.), etc., can be used as Sendai virus. Lipofectamine 3000 (Thermo Fisher SCIENTIFIC) can be used as a lipofection reagent. Epi5 Episomal iPSC Reprogramming Kit (Thermo Fisher SCIENTIFIC) can be used as the vector. As the “gelator” to pass through the gelator channel 233, either a combination of alginic acid and Ca-NTA or sodium hyaluronate can be used.

### Modification 5

The above embodiment is based on the premise that the first droplet generator 220, the second droplet generator 230, and the gel-encapsulated cell generator 240 are formed on a single baseplate 210. However, the embodiment is not limited thereto. For example, it is possible to form the first droplet generator 220, the second droplet generator 230, and the gel-encapsulated cell generator 240 on separate bases, respectively, and connect the three bases to form the baseplate 210. A baseplate may be formed for each of the other components.

### Modification 6

In the above embodiment, the stimulus for inducing gelation given by the stimulus introducer 243 is a gelator-dependent reagent. However, the embodiment is not limited thereto. The stimulus according to a modification 6 may be temperature or an external physical stimulus. In this case, the stimulus introducer 243 may be provided with a device for applying temperature or an external physical stimulus to the holding container 2432. A temperature adjuster can be used as a device for giving temperature. The external physical stimulus may be suitably selected from among light, an electric field, a magnetic field, impact, temperature, and the like according to the gelator.

### Generalization

According to the first embodiment described above, the gel-encapsulated cell production apparatus 200 has the first droplet generator 220, the second droplet generator 230, and the gel-encapsulated cell generator 240. The first droplet generator 220 generates a first droplet in which a processed cell is encapsulated. The second droplet generator 230 generates a second droplet in which a gelator is encapsulated. The gel-encapsulated cell generator 240 is connected to the first droplet generator 220 and the second droplet generator 230 via a channel, blends the first droplet and the second droplet, and generates a gel-encapsulated cell, which is the cell encapsulated in a gel.

According to the above configuration, a processed cell can be caused to gelate after cell processing is completed. Thus, it is possible to generate a gel-encapsulated cell without inhibiting cell processing due to the gelator. In addition, since it is possible to encapsulate a predetermined number of cells (typically, one cell) in one droplet to process the cell and cause the cell to gelate, the cell processing and the cell gelation can be implemented with high efficiency.

### Second Embodiment

Next, a cell culture system according to a second embodiment will be described below. In the description provided below, constituents having substantially the same functions as those of the first embodiment will be denoted by the same reference symbols as those used in the first embodiment, and a repeat description will be given only where necessary.

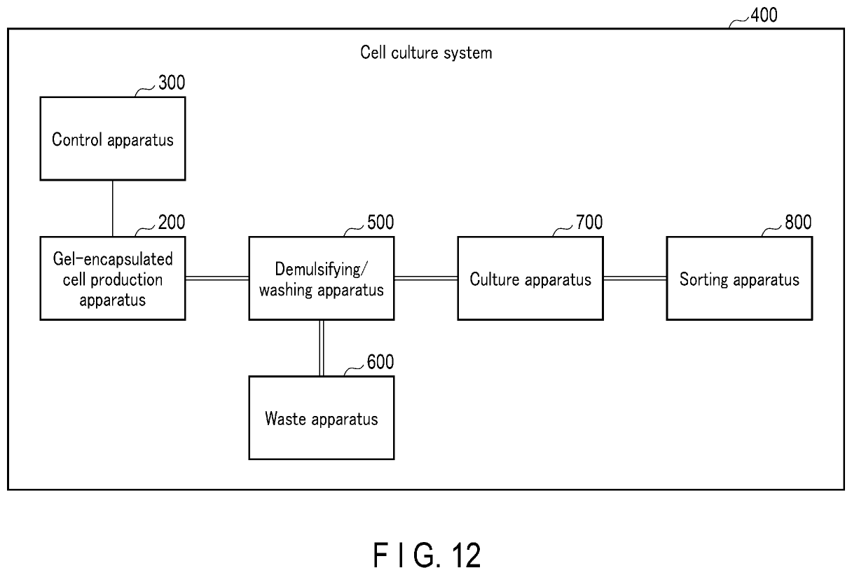

FIG. 12 shows an exemplary configuration of a cell culture system 400 according to the second embodiment. The cell culture system 400 is a mechanical system for producing a gel-encapsulated cell and culturing the gel-encapsulated cell. Specifically, the cell culture system 400 has a gel-encapsulated cell production apparatus 200, a control apparatus 300, a demulsification/washing apparatus 500, a waste apparatus 600, a culture apparatus 700, and a sorting apparatus 800. As an example, the gel-encapsulated cell production apparatus 200, the demulsification/washing apparatus 500, the culture apparatus 700, and the sorting apparatus 800 are connected in series to each other via channels, etc. The waste apparatus 600 is connected, in a branching manner, to the demulsification/washing apparatus 500 via a channel, etc.

The gel-encapsulated cell production apparatus 200 has a first droplet generator 220 that generates a first droplet in which a processed cell is encapsulated, a second droplet generator 230 that generates a second droplet in which a gelator is encapsulated, and a gel-encapsulated cell generator 240 that is connected to the first droplet generator 220 and the second droplet generator 230 via a channel, blends the first droplet and the second droplet, and encapsulates the cell in a gel. The first droplet generator 220 generates a first droplet by causing a first oil to react with a cell and a processing reagent for processing the cell. The second droplet generator 230 generates a second droplet by causing a second oil to react with a gelator. The gel-encapsulated cell generator 240 generates a gel-encapsulated cell, which is the cell encapsulated in a gel, by causing a third oil containing a gelation inducer to react with a processed cell contained in a blended droplet formed of the first droplet and the second droplet.

The control apparatus 300 is a machine that performs ejection and collection of various substances to and from the gel-encapsulated cell production apparatus 200, control of the devices, etc.

The demulsification/washing apparatus 500 is a machine arranged in the fore part of the culture apparatus 700. The demulsification/washing apparatus 500 washes the processed cell encapsulated in a gel in the gel-encapsulated cell. Specifically, the demulsification/washing apparatus 500 demulsifies the gel-encapsulated cell to remove the first oil, the second oil, and/or the third oil. The demulsification/washing apparatus 500 washes the cell with an aqueous solution, a non-aqueous solution, ultrasonic waves, etc., in the fore part and/or the rear part of the demulsification.

The waste apparatus 600 is a machine that disposes of the first oil, the second oil, and/or the third oil removed by the demulsification/washing apparatus 500. The waste apparatus 600 has an instrument for suctioning an oil and a tank for containing the suctioned oil.

The culture apparatus 700 is a machine that cultures the processed cell encapsulated in a gel in the gel-encapsulated cell. As an example, the culture apparatus 700 has a culture container that contains the gel-encapsulated cell, a sealed container that seals the culture container, a culture medium tank that stores a culture medium, a supplier that supplies the culture medium from the culture medium tank to the culture container, a temperature adjuster that adjusts the temperature inside the sealed container, an air adjuster that adjusts the concentration of various air components inside the sealed container, etc. A commercially available plate or microwell can be used as the culture container. Alternatively, a chamber of a microchannel having a structure that enables cell culture to be performed or a microchannel chip in which gel particles can be encapsulated may be used as the culture container.

The sorting apparatus 800 is a machine that selects a specific gel-encapsulated cell from among the cultured gel-encapsulated cells. For example, a cell sorter that sorts cells using magnetism or a laser can be used as the sorting apparatus 800. Alternatively, a microchannel chip that can sort gel-encapsulated cells based on their particle sizes, etc., may be used as the sorting apparatus 800.

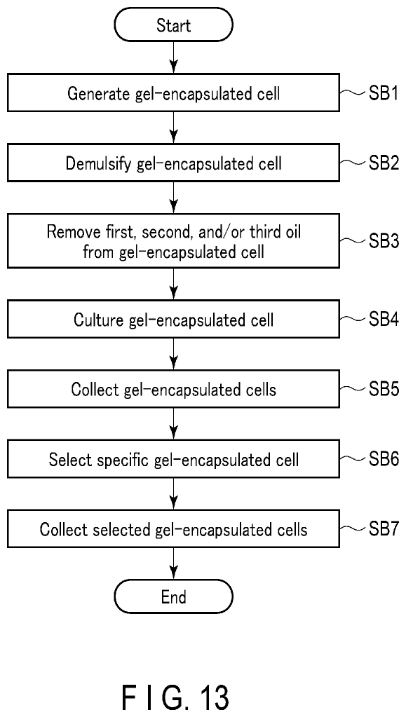

Next, a process of culturing a gel-encapsulated cell using the cell culture system 400 will be described with reference to FIG. 13. FIG. 13 shows a process procedure of culturing a gel-encapsulated cell using the cell culture system 400. As shown in FIG. 13, the gel-encapsulated cell production apparatus 200 generates a gel-encapsulated cell (step SB1). The gel-encapsulated cell may be generated according to the procedure described in the first embodiment.

After step SB1, the demulsification/washing apparatus 500 demulsifies the gel-encapsulated cell generated in step SB1 (step SB2). The demulsification of the gel-encapsulated cell separates the processed cell of the gel-encapsulated cell from the first, second, and/or third oil. The gel-encapsulated cell production apparatus 200 and the demulsification/washing apparatus 500 are connected to each other via a channel. The gel-encapsulated cell may be automatically supplied from the gel-encapsulated cell production apparatus 200 to the demulsification/washing apparatus 500. An operator may take the gel-encapsulated cell out of the gel-encapsulated cell production apparatus 200 and put it in the demulsification/washing apparatus 500.

After step SB2, the demulsification/washing apparatus 500 removes the first, second, and/or third oil from the gel-encapsulated cell demulsified in step SB2 (step SB3). The first, second, and/or third oil are removed by the waste apparatus 600.

After step SB3, the culture apparatus 700 cultures the gel-encapsulated cell from which the oil (s) has (have) been removed in step SB3 (step SB4). The demulsification/washing apparatus 500 and the culture apparatus 700 are connected to each other via a channel. The gel-encapsulated cell may be automatically supplied from the demulsification/washing apparatus 500 to the culture apparatus 700. An operator may take the gel-encapsulated cell out of the demulsification/washing apparatus 500 and moved it to the culture apparatus 700.

In step SB4, the culture apparatus 700 seeds the gel-encapsulated cell and cultures it under predetermined culture conditions. Since the processed cell is encapsulated in a gel, there is less influence of the surrounding cells and the environment, allowing for uniform cell growth. The gel is expected to also have a function as scaffolding. The first, second, and/or third oil have/has been removed from the gel-encapsulated cell in step SB3. Because of this, improved culture efficiency can be expected. Through the culturing, multiple cells are encapsulated in one gel particle.

After step SB4, the gel-encapsulated cells after the culturing performed in step SB4 are collected (step SB5). The gel-encapsulated cells may be collected by any collecting apparatus or by an operator.

After step SB5, the sorting apparatus 800 selects a specific gel-encapsulated cell from among the gel-encapsulated cells collected in step SB5 (step SB6). Since the cells are encapsulated in gels, the sorting apparatus 800 can make cell selection in units of cell. If different cells are cultured in units of gel-encapsulated cells or cultured under varying culture conditions, the same cells and the cells cultured under the same culture conditions can be easily selected.

The selection of a specific gel-encapsulated cell may be performed with an operator's hand without relying on the sorting apparatus 800. As an example, an operator may observe the gel-encapsulated cells with a microscope and select a specific gel-encapsulated cell. As another example, an operator may add a labeling reagent to the gel-encapsulated cells and select a specific gel-encapsulated cell according to the signal of the labeling reagent.

After step SB6, the selected gel-encapsulated cells are collected (step SB7). The gel-encapsulated cells may be collected by any collecting apparatus or by an operator.

The explanation of the process of culturing a gel-encapsulated cell shown in FIG. 13 will end with the above descriptions. The flow of the process of culturing a gel-encapsulated cell shown in FIG. 13 is an example, and the embodiment is not limited thereto. Various modifications can be made, provided that the gel-encapsulated cell can be cultured.

### Modification 7

The cell culture system 400 has the gel-encapsulated cell production apparatus 200, the control apparatus 300, the demulsification/washing apparatus 500, the waste apparatus 600, the culture apparatus 700, and the sorting apparatus 800; however, the embodiment is not limited thereto. The cell culture system 400 according to a modification 7 need not have the demulsification/washing apparatus 500 or the waste apparatus 600 if, for example, the first, second, and third oils need not be removed. As another example, the cell culture system 400 according to the modification 7 need not have the sorting apparatus 800 if gel-encapsulated cells need not be sorted.

### Generalization

According to the second embodiment described above, the cell culture system 400 has the gel-encapsulated cell production apparatus 200 and the culture apparatus 700. The gel-encapsulated cell production apparatus 200 has the first droplet generator 220, the second droplet generator 230, and the gel-encapsulated cell generator 240. The first droplet generator 220 generates a first droplet in which a processed cell is encapsulated. The second droplet generator 230 generates a second droplet in which a gelator is encapsulated. The gel-encapsulated cell generator 240 is connected to the first droplet generator 220 and the second droplet generator 230 via a channel, blends the first droplet and the second droplet, and generates a gel-encapsulated cell, which is the cell encapsulated in a gel. The culture apparatus 700 cultures the cell encapsulated in a gel.

According to the above configuration, it is possible to culture a processed cell for each droplet. If one processed cell is included in one droplet, culturing can be performed for each individual cell. Also, providing the cell culture system 400 with the sorting apparatus 800 makes it possible to easily sort a colony of a population of single cells included in gels. In addition, according to the second embodiment, these steps can be performed consistently with a single mechanical system; therefore, procedural variation can be suppressed, and cell treatment can be easily performed.

According to at least one of the embodiments described above, a processed cell encapsulated in a gel can be easily produced with a high quality.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A gel-encapsulated cell production apparatus comprising:
a first droplet generator configured to generate a first droplet in which a processed cell is encapsulated;
a second droplet generator configured to generate a second droplet in which a gelator is encapsulated; and
a gel-encapsulated cell generator connected to the first droplet generator and the second droplet generator via a channel and configured to blend the first droplet and the second droplet and generate a gel-encapsulated cell, the gel-encapsulated cell being the cell encapsulated in a gel originating in the gelator.

2. The gel-encapsulated cell production apparatus according to claim 1, wherein the first droplet generator mixes the cell and a processing reagent for processing the cell and generates the first droplet.

3. The gel-encapsulated cell production apparatus according to claim 2, wherein the first droplet generator includes:
a first channel through which the cell passes;
a second channel through which the processing reagent passes;
a third channel through which a first oil passes;
a generator where the first channel, the second channel and the third channel meet, the generator being configured to generate a third droplet using the first oil, the cell and the processing reagent being encapsulated in the third droplet; and
a promoter configured to promote processing of the cell inside the third droplet using the processing reagent and generate the first droplet.

4. The gel-encapsulated cell production apparatus according to claim 3, wherein the promoter includes a processing promoting container being a container for holding the third droplet and adjusted to have an optimal temperature by a temperature adjuster.

5. The gel-encapsulated cell production apparatus according to claim 3, wherein the promoter includes a channel having a width smaller than a particle size of the third droplet.

6. The gel-encapsulated cell production apparatus according to claim 5, wherein the second droplet generator includes:
a fifth channel through which the gelator passes;
a sixth channel through which a second oil passes;
a seventh channel configured to combine the fifth channel and the sixth channel and generate the second droplet using the second oil, the gelator being encapsulated in the second droplet.

7. The gel-encapsulated cell production apparatus according to claim 1, wherein the gel-encapsulated cell generator includes:
a blender configured to blend the first droplet and the second droplet to generate a blended droplet; and
a stimulus introducer configured to give the blended droplet a stimulus for inducing gelation of the cell with the gelator.

8. The gel-encapsulated cell production apparatus according to claim 7, wherein the blender includes:
a holding container for holding the first droplet and the second droplet; and
an electrode for applying, to the first droplet and the second droplet held in the holding container, an electric field for inducing blending of the first droplet and the second droplet.

9. The gel-encapsulated cell production apparatus according to claim 7, wherein the gel-encapsulated cell generator further comprises a stirrer arranged between the blender and the stimulus introducer and having a stirring channel for stirring the gelator and the cell inside the blended droplet.

10. The gel-encapsulated cell production apparatus according to claim 9, wherein
the stimulus is a reagent that includes a crosslinker, an acid, or an ion according to the gelator, and
the stimulus introducer is connected to the stirring channel and includes a holding container for holding the blended droplet and a third oil containing the reagent.

11. The gel-encapsulated cell production apparatus according to claim 9, wherein
the stimulus is temperature or an external physical stimulus, and
the stimulus introducer applies the temperature or the external physical stimulus to the holding container.

12. The gel-encapsulated cell production apparatus according to claim 1, wherein
the cell is a PBMC, a hematopoietic stem cell, an umbilical cord blood cell, a skin cell, or a fibroblast,
a processing reagent for processing the cell is either Sendai virus or a combination of a lipofection reagent and a vector, and
the gelator is either a combination of alginic acid and Ca-NTA or sodium hyaluronate.

13. The gel-encapsulated cell production apparatus according to claim 1, further comprising a baseplate on which the first droplet generator, the second droplet generator, and the gel-encapsulated cell generator are formed.

14. A cell culture system comprising:
a gel-encapsulated cell production apparatus comprising:
a first droplet generator configured to generate a first droplet in which a processed cell is encapsulated;
a second droplet generator configured to generate a second droplet in which a gelator is encapsulated; and
a gel-encapsulated cell generator connected to the first droplet generator and the second droplet generator via a channel and configured to blend the first droplet and the second droplet and generate a gel-encapsulated cell, the gel-encapsulated cell being the cell encapsulated in a gel originating in the gelator; and
a culturing unit configured to culture the cell encapsulated in the gel.

15. The cell culture system according to claim 14, further comprising a washer arranged in a fore part of the culturing unit and configured to wash the cell encapsulated in the gel, wherein
the first droplet generator generates the first droplet by causing a first oil to react with the cell and a processing reagent for processing the cell,
the second droplet generator generates the second droplet by causing a second oil to react with the gelator,
the gel-encapsulated cell generator generates a gel-encapsulated cell by causing a third oil containing a gelation inducer to react with a processed cell contained in a blended droplet composed of the first droplet and the second droplet, the gel-encapsulated cell being the cell encapsulated in the gel,
the washer demulsifies the gel-encapsulated cell and removes the first oil, the second oil, and/or the third oil, and
the culturing unit cultures the cell encapsulated in the gel, the first oil, the second oil, and/or the third oil being removed from the cell.

16. The cell culture system according to claim 14, further comprising a sorter configured to select a specific cell from among cells obtained by culturing the cell.
